(19) United States
(12) Patent Application Publication
(10) Pub. No.: US 2017/0350894 A1
(43) Pub. Date: Dec. 7, 2017

(54) Method of diagnosing and monitoring bladder cancer

(71) Applicant: Francis Eugene Wilkinson, Frankfort, IN (US)

(72) Inventor: Francis Eugene Wilkinson, Frankfort, IN (US)

(21) Appl. No.: 15/684,229

(22) Filed: Aug. 23, 2017

Related U.S. Application Data
(62) Division of application No. 14/970,972, filed on Dec. 16, 2015.

(51) Int. Cl. G01N 33/574

(52) U.S. Cl. CPC G01N 33/57407; G01N 33/5743; G01N 2333/705; G01N 2800/56; G01N 2333/90245

(57) ABSTRACT

The present invention provides a method for the diagnosing and monitoring of bladder cancer.

This is a Divisional Application from application Ser. No. 14/970,972 which was submitted Dec. 15, 2015.

### FIELD OF THE INVENTION

This invention relates, in part, to newly developed assays for diagnosing and monitoring bladder cancer.

### BACKGROUND OF THE INVENTION

The American Cancer Society estimated that 74,000 people will be diagnosed with bladder cancer and that 16,000 people will die of the disease in the United States in 2015. There are more than 500,000 people in the United States who have been diagnosed with bladder cancer and are living with the disease. See “Bladder Cancer.” American Cancer Society p. 5(Feb. 25, 2015). Men die of bladder cancer at more than three times the rate as women. See Howlader et al., “Cancer of the Urinary Bladder (Invasive and In Situ).” SEER Cancer Statistics Review, 1975-2012 Section 27 p. 6 (April 2015).

A large majority of the cases of bladder cancer are identified while the tumor is either in situ or localized, 51% and 35%, respectively. Seven per cent of the cases are diagnosed as regional, four percent as distant, and three percent are diagnosed as unstaged. Those diagnosed with in situ disease have a five year survival of 95.9%. Those with localized, regional and distant diagnosis have five-year survival rates of 69.9%, 34.0% and 5.4%, respectively. See Howlader et al., “Cancer of the Urinary Bladder (Invasive and In situ).” SEER Cancer Statistics Review, Section 27 1975-2012 p. 8 (April 2015).

Bladder cancer develops in the epithelial layer. The bladder epithelial membrane that makes contact with urine is distinctive as it is dense when visualized by electron microscopy and is twice as thick as the other membranes of bladder epithelium. This membrane is called the Asymmetric Unit Membrane, or AUM. The AUM is critical in maintaining the structural integrity of the bladder from the increase in hydrostatic pressure during the micturition cycle and serves as a protective barrier to surrounding tissues from toxins in urine.

It was found that the extra thickness and unusual appearance of the AUM is due to a layer of protein. The proteins comprising the AUM were determined to be exclusively the uroplakins: uroplakin I, uroplakin II, and uroplakin III. See Wu et al., J. Biol. Chem. 265.31(1990):19170-19179. Further research showed that Uroplakin I is actually two similar proteins: Uroplakin Ia and Uroplakin Ib. See Yu et al., J. Cell Biol. 125.1(1994):171-182. The assembly of the uroplakins into the AUM proceeds by UPK 1a associating with UPK II, and UPK 1b associating with UPK III to form heterodimers. Both heterodimers join together into a larger subunit, two of which comprise the basic subunit. See Wu et al., Kidney Int. 75.11(2009):1153-1165.

The bladder epithelium is a dynamic structure with the uroplakins being continuously added to the bladder as the volume of urine gradually increases. The uroplakins are removed during the remodeling process as well as when the volume of the urine rapidly decreases. The uroplakins are not recycled, but are transported to the lysosomes, where they are degraded. The individual proteins involved in the process are being determined. See Guo et al., Traffic 10.9(2009)1350-1361. See Vieira et al., PLoS One 9.6(2014):1-15.

The relationship of the uroplakins with bladder cancer in humans was initially investigated in 1995 using polyclonal antibodies generated against bovine AUM. The antibodies used in this study reacted primarily with uroplakin III. Immmuohistochemistry was performed on paraffin blocks of normal and malignant tissues. Positive reactions were found in fourteen of sixteen (88%) papillary noninvasive transitional cell carcinoma, TCC, twenty-nine of fifty-five (53%) invasive TCCs, and twenty-three of thirty-five (66%) TCC with metastases. No positive reactions were found with any of the 177 biopsy samples from other cancers. See Moll et al., Am. J. Pathol. 147.5(1995):1383-1397. The initial results were extended using the same pan-uroplakin antibodies used in the previous report. This immunohistochemical study was performed on tissue arrays from bladder cancer tumor tissues, and the normal controls were from patients who had undergone a cystectomy for reasons other than genitourinary malignancy. The uroplakins were found in nine of the nine (100%) normal controls. Of patients with non-muscle-invasive urothelial carcinomas on transurethral resection, sixty-three of seventy-four (85%) were positive; of patients who underwent radical cystectomy for advanced urothelial carcinomas 104 of 202 (51.5%) were positive; and of patients with lymph node metastases thirty-three of fifty (66%) were positive. See Huang et al., Hum. Path. 38.11(2007):1703-1713. Rabbit antibodies generated against a synthetic peptide of bovine UP II and against total bovine uroplakins showed uroplakin reactivity in seventeen of forty-three (39%) cases of conventional TCC. See Wu et al., Cancer Res. 58.6(1998):1291-1297.

A rabbit antibody generated against a peptide of human UPK Ia was found to be excellent in the immunohistochemical staining of cancerous urothelial tissue. This antibody stained greater than ten per cent of the cells in sixty-one of sixty-three primary lesions (96.8%). Depending on pathological grade, high expression (50% or more positive cancer cells) was observed in seventeen of eighteen (94.4%) moderately- to well-differentiated TCC and in thirty-six of forty-five (80%) poorly differentiated TCC. With regard to tumor invasion, high expression was noted in twenty of twenty-two (90.9%) superficial and thirty-three of forty-one (80.5%) muscle-invasive TCC. See Kageyama et al., Jpn. J. Cancer Res. 93.5(2002):523-531.

A monoclonal antibody to uroplakin II was shown to be superior to a monoclonal antibody to uroplakin III in staining urothelial cancer tissue. UPK II and UPK III stained 78.5% and 55.3% of fifty-six cases of the carcinoma of the bladder, respectively. The UPK II antibody was also used to determine staining of the standard FDA panel of thirty-seven normal tissues. The antibody stained only bladder and ureter of the normal tissues. See Hoang et al., Arch. Pathol. Lab. Med. 138.7(2014):943-949. Another group found that uroplakin III antibody stained sixty-four of 112 paraffin-embedded urothelial tumors (57.1%). Tissue microarray analysis showed no uroplakin III immunoreactivity in tissue cores of non-urothelial tumors. See Parker et al., Am. J. Surg. Pathol. 27.1(2003):1-10.

Gene expression profiles in normal tissue showed that each of the uroplakins is expressed only in the urothelium. In seven cases of non-invasive bladder tumors (TCC) the UPK Ia, UPK Ib, and UPK II genes were expressed in six, seven and seven of the cases, respectively. All four of the uroplakins were expressed in RT4 cells, which are derived from a Grade 1 TCC. See Lobban et al., Am. J. Pathol. 153.6(1998):1957-1967. In a subsequent study all four uroplakins were expressed by the urothelium, but UPK Ib was detected in several non-urothelial tissues, and UPK III was expressed by prostatic glandular epithelium. UPK Ia and UPK II appeared to be urothelium-specific. Ten cases of TCC tested all expressed one or more of the uroplakins, but none of the cases expressed all four of the uroplakins. Eighty per cent of the primary tumors expressed at least one uroplakin and that seventy per cent of the lymph nodes of patients with metastatic disease expressed at least one of the uroplakins. See Olsburgh et al., J. Pathol. 199.1(2003):41-49.

A study of the expression of the uroplakins in circulating blood cells of bladder cancer patients found UPK II mRNA in the blood of three of ten metastatic bladder cancer patients, but no UPK II mRNA was found in the blood of normal individuals or in any of fifty cases of non-metastatic TCC. See Li et al., J. Urol. 162.3(1999):931-935. The implication of finding uroplakin expressing cells in the circulation is that such cells have broken away from the primary tumor in the bladder and are the cells that travel to other sites in the body and start the secondary tumor.

Yuasa and collaborators developed an assay that could detect one HT1197 cell, a TCC cell line, when added to 5 ml of peripheral blood. UPK Ib and UPK III mRNA were found in the peripheral blood of all three patients tested who have metastatic TCC but not in the blood of any of the nine patients with non-metastatic TCC or in the blood of three healthy volunteers. See Yuasa et al., Jpn. J. Cancer Res. 89.9(1998):879-882.

Nested primers were used to study uroplakin gene expression because nested primers reduce unexpected gene products. Yuasa and collaborators added nested primers to their assay mentioned above. This assay could also detect a single HT1197 cell added to 5 ml of peripheral blood. The UPK II gene was expressed in the peripheral blood from all three patients with metastatic TCC, but not from the blood of any of nine patients with non-metastatic TCC, nor in the blood of three healthy volunteers. See Yuasa et al., Int. J. Urol. 6.6(1999):286-292.

UPK II expression using nested primers in circulating cells was found to increase as the pathological stage of bladder cancer increased. UPK II mRNA-positive cells were detected in 3 of 29 (10.3%) patients with superficial cancers (pTa-1N0M0), four of fourteen (26.8%) patients with muscularly invasive cancers (pT2-4N0M0), 2 of 5 (40.0%) loco-regional node-positive patients (pN1-2M0), and six of eight (75.0%) patients with distant metastases. In addition, sequential blood sampling was performed in three patients during and after systemic chemotherapy, and UPK II-positive cells were found to have disappeared in two patients who responded well to the systemic chemotherapy demonstrating. This latter result suggests the possible utility of UPK II expression as a monitoring marker. See Lu et al., Clin. Cancer Res. 6.8(2000):3166-3171.

It was found that UPK II mRNA in cells circulating in the blood from fifteen bladder cancer patients (58%) with a stage of pT2 or advanced stages. UPK II expression was found in the blood of twenty-five of 108 (23%) patients diagnosed with TCC. UPK II expression was also found in the circulating cells of five patients (8%) with superficial disease (pTa and pT1), and thirteen (20%) and 10 (56%) for patients with grades 2 and 3 respectively. No UPK II expression was found in the circulating cells of twenty healthy volunteers. See Okegawa et al., J. Urol. 171.4(2004):1461-1466.

UPK II gene expression in lymph nodes of bladder cancer patients was shown to have promise in identifying patients thought to have localized disease but who actually have metastatic bladder cancer. Sixty-six pelvic lymph nodes from patients diagnosed as not having metastases were tested, and six (9%) of them were found to express UPK II. See Wu et al., J. Urol. 174.6(2005):2138-2142.

The expression of UPK II in lymph nodes was shown to be an excellent predictor of disease recurrence in a group of patients who had undergone a standard radical cystectomy and pelvic lymph node dissection. Nineteen of twenty-four (79%) of patients with node positive UPK II expression had a recurrence of bladder cancer during four plus years of follow-up with a p of <0.0001. Of eleven patients who were pathologically lymph node negative and UPK II positive, ten (90.9%) had a recurrence. Only one of twenty-two (4.5%) patients who was node negative for UPK II expression had recurrence. See Copp et al., Cancer. 107.1(2006):60-66.

In contrast to the preceding data, UPK II expression was found in several normal tissues that are not part of the urinary tract. UPK II expression was also found in circulating cells of individuals with lung cancer as well as pancreatic cancer. See Hirata et al., Oncol. Rep. 10.4(2003):963-966. This report can be discounted because none of the authors has been an author on any other report of bladder cancer and the uroplakins. In addition, the journal, Oncology Reports, is not included among the journals in PubMed of the National Library of Medicine.

Elevated serum levels of UPK III were reported to correlate highly with pathological grade, pathological stage, and muscle invasion relative to non-muscle invasion of bladder cancer. A mouse antibody to human UPK III and an anti-mouse IgG conjugated with horseradish peroxidase were used to quantify UPK III in a dot blot system. See Tsumura et al., Asian Pac. J. Cancer Prev. 16.4(2015):1539-1543. The results are suspect because the analyses were performed using a dot blot with the “background intensity around the spot,” being the control value. A true comparison is to treat the samples of serum on the dot blot membranes with the UPK III antibody as the experimental and the control is similarly treated, but without the antibody.

This summary of the literature has shown that the association of the uroplakins with bladder cancer has increased as the technology and reagents to study the relationship have improved to the point that it is now clear that wherever there are bladder cancer cells, that the protein and mRNA of all four of the uroplakins are present.

Therefore it would be expected that if the blood level of any one of the uroplakins correlates with bladder cancer, that there is also a positive correlation of the blood level of the other three uroplakins with bladder cancer.

There are at least three medical conditions that present a pressing need for sensitive diagnostic tests for bladder cancer. These include:
1. As an early detection marker. The purpose of early detection markers is to detect the cancer while it is localized. Localized tumors are generally small and usually can be surgically removed before the cancer metastasizes. Cancer that has metastasized, even to nearby organs, is considered to be incurable.
The PSA test for prostate cancer is the only FDA-approved early detection marker for any cancer. The PSA test has revolutionized the diagnosis of prostate cancer and has been instrumental in lowering the age-adjusted death rate by fifty per cent since 1992. See Howlader et al., “Cancer of the Prostate (Invasive).” Section 23 SEER Cancer Statistics Review, 1975-2012, p. 6 (April 2015). The PSA test has an actual clinical sensitivity of 53.2% and 69.9% for stages A and B, respectively. This indicates that the PSA test cannot identify all cases of early stage prostate cancer. See “Architect System Total PSA.” Abbott Laboratories p. 6 (June 2007).
The introduction of an early detection marker for bladder cancer could reduce the deaths due to bladder cancer by 10,000 per year.
2. As a staging marker. Staging markers assist clinicians in determining whether the cancer is localized or has metastasized. The five year survival data of bladder cancer patients diagnosed with localized disease illustrates the problem as 30.1% of these patients die within five years of diagnosis. See Howlader et al., “Cancer of the Urinary Bladder (Invasive and In Situ).” Section 23 SEER Cancer Statistics Review, 1975-2012, p. 8 (April 2015). This data suggests that many of these bladder cancer patients actually had metastatic disease when the initial diagnosis was that they had localized disease. The treatments for localized and metastatic disease are quite different, and proper staging is critical to a successful treatment.
3. As a monitoring marker. Monitoring markers are a real time analytic of the growth status of previously diagnosed cancer. Monitoring markers are used to determine if the cancer shows no evidence of disease, is stable, is responding to treatment, or is progressing. The test result can be critical in determining the proper treatment.
   There are no tests for the early detection of bladder cancer. There are four urine tests that are used for the monitoring of bladder cancer: UroVysion, BTA, Immunocyt, and NMP22 BladderChek. See “Bladder Cancer.” American Cancer Society p. 12 (Feb. 25, 2015).

### DETAILED DESCRIPTION OF CERTAIN EMBODIMENTS OF THE INVENTION

The present invention relates to diagnostic assays, both quantitative and qualitative for detecting levels of the uroplakins or their polypeptides in bodily fluids including empirical determination of normal and abnormal levels. Thus, for instance, a diagnostic assay in accordance with the invention of detecting over-expression of the uroplakins in bodily fluids of individuals who may have bladder cancer compared to levels in healthy individuals may be used to detect the presence of bladder cancer. Further, the present method of quantifying the level of the uroplakins is particularly useful for discriminating between bladder cancer and other diseases of the bladder since some of the other methods for discriminating between bladder cancer and other diseases of the bladder require a biopsy and examination by a pathologist. The current invention could be performed in a physician's office and have a reliable result within three hours. Assay techniques that can be used to determine levels of gene expression of antigens like the uroplakins, of the present invention, in a sample derived from a host are well-known to those of skill in the art. Such assay methods include, but are not limited to, radioimmunoassay, competitive binding assays, western blot analyses, dot blots, and ELISAs.

Without limiting the type of assay in the instant invention, ideally for a quantitative diagnostic assay a positive result indicating the disease is one in which blood levels are higher than three standard deviations above the mean level for a normal healthy population of individuals (99.865% of the population). A best case result would yield 100% clinical sensitivity and 100% clinical specificity, which means that the test correctly identifies every individual with the disease as having the disease and also identifying 100% of those who do not as not having the disease, respectively.

The present invention is further described by the following examples. These examples are provided solely to illustrate the invention by reference to specific embodiments. These examples, while illustrating certain specific aspects of the invention, do not portray the limitations or circumscribe the scope of the disclosed invention.

### BRIEF DESCRIPTION OF THE SEVERAL VIEWS OF THE DRAWING

Example 1 Demonstrating a positive correlation between bladder cancer and the blood level of UPK II.

FIG. 1, is a dot blot comparing the plasma level of UPK II of a woman with Stage IV bladder versus that of three dilutions of plasma from a healthy sixty-five year old man.

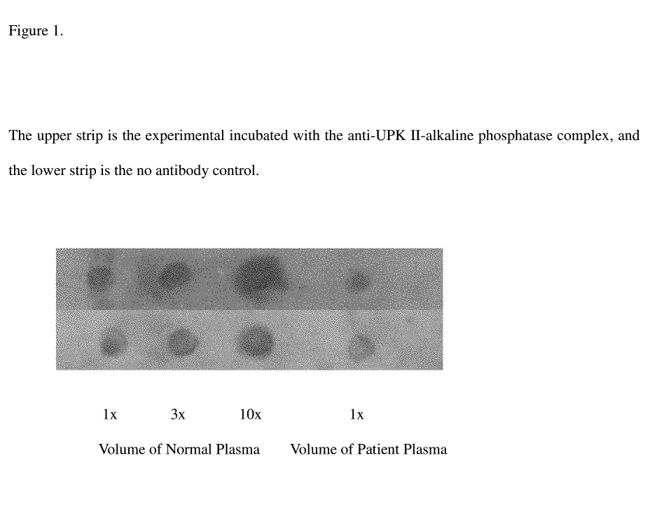

The normal plasma was from a healthy adult White male. The patient plasma was from a 59 year old White female who was initially diagnosed with localized bladder cancer. The patient had a complete cystectomy twenty months prior to the collection of the plasma sample. The patient was later diagnosed as Stage IV when lung metastases were detected, which was prior to the collection of the sample. Based upon the plasma half-life of human serum albumin, which is twenty-one days, the blood level of uroplakin II had undergone 28.5 half lives and would be reduced to less than two hundred millionth its pre-operative level. See Anderson et al., Mol. Cell. Proteomics 1.11(2002):845-867. Even if the half-life of UPK II in plasma were four times as long as that of albumin, the UPK II blood level would have been reduced to less than one per cent of its pre-operative level.

A small volume of diluted patient plasma and 1×, 3× and 10× volumes of the normal plasma, relative to the bladder cancer patient plasma, were spotted on two strips of nitrocellulose. The other binding sites on the nitrocellulose were covered by incubating the nitrocellulose in a solution of bovine serum albumin.

A mouse monoclonal antibody generated against a peptide of human Uroplakin II was obtained from a commercial vendor. This antibody has been shown to react exclusively with normal urothelium or urothelium-derived cancer tissue. See Hoang et al., Arch. Pathol. Lab. Med. 138.7(2014):943-949. The antibody was biotinylated and reacted with streptavidin Alkaline-Phosphatase to produce an antibody with the reporter Alkaline-Phosphatase attached. The experimental and control strips were put into incubation media with or without the anti-UPK II Alkaline Phosphatase complex, respectively. Both were washed, then put into buffer containing the substrate for alkaline phosphatase to visualize the UPK II.

Both strips of nitrocellulose show positive reactions in nearly all spots including the controls. Reaction product in the no antibody controls is due to a component of human plasma that reacts with the substrate for alkaline phosphatase. The plasma from the healthy male had much more of this component than the patient plasma, which was nearly zero.

The normal plasma shows a non-linear increase in UPK II with the amount of plasma analyzed.

The relative quantity of UPK II in the patient plasma cannot be determined exactly, but it is greater than the 1× volume and less than the 10× volume of Normal Plasma. Regardless of the amount of UPK II in the patient plasma relative to the healthy individual, the amount is much greater than the nearly zero quantity that would be expected twenty months after the UPK II producing tissues had been surgically removed from the patient.

Since UPK II and the other uroplakins are synthesized only by urothelial cells, the UPK II in the patient plasma must have been synthesized by the metastases of the bladder cancer.

These results indicate that the level of and of the four uroplakin proteins in blood and other human bodily fluid could be used in diagnostic assays for the diagnosing and monitoring of bladder cancer.

### REFERENCES

Anderson et al., “The Human Plasma Proteome History, Character, and Diagnostic Prospects.”Mol. Cell. Proteomics 1.11(2002):845-867.

“Architect System Total PSA.” pp. 1-8 Abbott Laboratories (June 2007).

“Bladder Cancer.” pp. 1-58 The American Cancer Society (Feb. 25, 2015).

Copp et al., “Prospective Evaluation of the Prognostic Relevance of Molecular Staging for Urothelial Carcinoma.”Cancer. 107.1(2006):60-66.

Guo et al., “Involvement of Vps33a in the Fusion of Uroplakin-Degrading Multivesicular Bodies with Lysosomes.” 10.9(2009)1350-1361.

Hirata et al., “Genetic Detection for Hematogenous Micrometastasis in Patients with Various Types of Malignant Tumors Using Uroplakin II Derived Primers in Polymerase Chain Reaction.” Oncol. Rep. 10.4(2003):963-966. Pubmed Abstract, PMID 12792753.

Hoang et al., “A Newly Developed Uroplakin II Antibody with Increased Sensitivity in Urothelial Carcinoma of the Bladder.” Arch. Pathol. Lab. Med. 138.7(2014):943-949.

Howlader et al., “Cancer of the Prostate (Invasive).” SEER Cancer Statistics Review, 1975-2012, Section 23 pp. 1-22 National Cancer Institute. Bethesda, Md. http://seer.cancer.gov/csr/19752012/, based on November 2014 SEER data submission, posted to the SEER web site, April 2015.

Howlader et al., “Cancer of the Urinary Bladder Section 27 (Invasive and In situ).” SEER Cancer Statistics Review, 1975-2012, Section 27 pp. 1-26 National Cancer Institute. Bethesda, Md., http://seer.cancer.gov/csr/19752012/, based on November 2014 SEER data submission, posted to the SEER web site, April 2015.

Huang et al., ‘Persistent Uroplakin Expression in Advanced Urothelial Carcinomas: Implications in Urothelial Tumor Progression and Clinical Outcome.” Hum. Path. 38.11(2007):1703-1713.

Kageyama et al., “High Expression of Human Uroplakinla in Urinary Bladder Transitional Cell Carcinoma.” Jpn. J. Cancer Res. 93.5(2002):523-531. Pubmed Abstract, PMID 12036448.

Li et al., “Detection of Circulating Uroplakin-Positive Cells in Patients with Transitional Cell Carcinoma of the Bladder.” J. Urol. 162.3(1999):931-935. Pubmed Abstract, PMID 10458411.

Lobban et al., “Uroplakin Gene Expression by Normal and Neoplastic Human Urothelium.” Am. J. Pathol. 153.6(1998):1957-1967.

Lu et al., “Detection of Circulating Cancer Cells by Reverse Transcription-Polymerase Chain Reaction for Uroplakin II in Peripheral Blood of Patients with Urothelial Cancer.” Clin. Cancer Res. 6.8(2000):3166-3171.

Moll et al., “Uroplakins, Specific Membrane Proteins of Urothelial Umbrella Cells, as Histological Markers of Metastatic Transitional Cell Carcinomas.” Am. J. Pathol. 147.5(1995):1383-1397.

Okegawa et al., “Value of Reverse Transcription Polymerase Chain Assay in Peripheral Blood of Patients with Urothelial Cancer.” J. Urol. 171.4(2004):1461-1466. Pubmed Abstract, PMID 15017198.

Olsburgh et al., “Uroplakin Gene Expression in Normal Human Tissues and Locally Advanced Bladder Cancer.” J. Pathol. 199.1(2003):41-49. Pubmed Abstract, PMID 12474225.

Parker et al., “Potential utility of uroplakin III, thrombomodulin, high molecular weight cytokeratin, and cytokeratin 20 in noninvasive, invasive, and metastatic urothelial (transitional cell) carcinomas,” Am. J. Surg. Pathol. 27.1(2003):1-10. Pubmed Abstract, PMID 12502922.

Tsumura et al., “High Expression Level of Preoperative Serum Uroplakin III is Associated with Biologically Aggressive Bladder Cancer.” Asian Pac. J. Cancer Prey. 16.4(2015):1539-1543.

Vieira et al., “SNX31: A Novel Sorting Nexin Associated with the Uroplakin-Degrading Multivesicular Bodies in Terminally Differentiated Urothelial Cells.” PLoS One 9.6(2014):1-15.

Wu et al., “Large Scale Purification and Immunolocalization of Bovine Uroplakins I, II, and III.” J. Biol. Chem. 265.31(1990):19170-19179.

Wu et al., “Uroplakin II Gene Is Expressed in Transitional Cell Carcinoma But Not in Bilharzial Bladder Squamous Cell Carcinoma: Alternative Pathways of Bladder Epithelial Differentiation and Tumor Formation.” Cancer Res. 58.6(1998):1291-1297.

Wu et al., “Uroplakin II as a promising marker for molecular diagnosis of nodal metastases from bladder cancer: comparison with cytokeratin 20.” J. Urol. 174.6(2005):2138-2142. Pubmed Abstract, PMID 16280744.

Wu et al., ‘Uroplakins in Urothelial Biology, Function, and Disease.” Kidney Int. 75.11(2009):1153-1165.

Yu et al., “Uroplakins Ia and Ib, Two Major Differentiation Products of Bladder Epithelium, Belong to a Family of Four Transmembrane Domain (4TM) Proteins.” J. Cell Biol. 125.1(1994):171-182.

Yuasa et al., “Expression of Uroplakin Ib and Uroplakin III Genes in Tissues and Peripheral Blood of Patients with Transitional Cell Carcinoma.”Jpn. J. Cancer Res. 89.9(1998):879-882.

Yuasa et al., ‘Expression of transitional cell-specific genes, uroplakin Ia and II, in bladder cancer: Detection of circulating cancer cells in the peripheral blood of metastatic patients.” Int. J. Urol. 6.6(1999):286-292.

1. A method for diagnosing the presence of bladder cancer, the method comprising: analyzing human blood, plasma, serum, tears, sweat, vaginal discharge, saliva or urine from the subject at risk for developing bladder cancer with an assay that specifically detects any of the uroplakins in the sample.

2. (canceled)

3. The method of claim 1 in which the uroplakins are: Uroplakin Ia, Uroplakin Ib, Uroplakin II, or Uroplakin III.

4. The method of claim 1 wherein the uroplakin is detected by an analytical method such as, but not limited to, ELISA, radioimmunoassay, competitive binding assay, Western blot, or dot blot.

5. The method of claim 1 wherein the level of the uroplakin above a cutoff is indicative of the presence of bladder cancer in an individual suspected of having bladder cancer.
